(19) United States
(12) Patent Application Publication
(10) Pub. No.: US 2002/0138024 A1
(43) Pub. Date: Sep. 26, 2002

(54) Statement regarding federally sponsored research or development

(76) Inventor: Olarewaju James Oladipo, Canton, MA (US)

Correspondence Address: OLADIPO OLAREWAJU JAMES, P.O. Box 446, CANTON, MA, 02021, US

(21) Appl. No.: 09/815,151

(22) Filed: Mar. 22, 2001

(51) Int. Cl. A61F 5/00

(52) U.S. Cl. 602/3

(57) ABSTRACT

The orthopedic cast decorative and instructional adhesive patch and tape having an exterior surface bearing imprinted and/or die cut perforations depicting graphical designs and patient care orders, and an inner surface coated with adhesives of the nature that will adhere permanently or releasably to the outer surfaces of polymeric and non-polymeric casting materials. The orthopedic cast decorative adhesive laminate, tape, or fabric having an optional liner that can be removed before application on the surface of the cast, or treated on the non-adhesive bearing surface with a coating enabling the tape to be wound into a roll. The orthopedic cast decorative and instructional adhesive patch and tape can be applied either as multiple patches bearing one or more graphical designs and instructions, or in a continuous form to cover all or part of applied cast.

### BACKGROUND OF INVENTION

While the methods, compositions and materials presented herein may be used in a variety of applications, they were specifically developed with respect to orthopedic casting tapes, instructional and decorative adhesive patches for application to such casting tapes. Therefore as background, relevant details relating to orthopedic casting tapes and problems overcome by this invention are provided. This invention relates to two aspects of use relating to orthopedic casting tapes. Firstly, the need to have a means to apply a decorative tape that will adhere without wrinkle and distortion of applied graphics to the surface of an applied cast, and without obliterating the pores on the cast surface. Secondly, the need for instructional patches bearing care instructions and, or decorative graphics applied to the surface of an applied cast without distortion of graphics and of a thickness that it would uniformly adhere, without wrinkle and could be easily removed and repositioned as desired.

Orthopedic casting tapes in use are currently of two types namely, Plaster of Paris, and polymeric casting materials. Polymeric casting tapes have gained widespread acceptance because of their added strength, light weight, and durability. These casting tapes undergo a water activated process that results in hardening of the material when soaked in water and applied to an injured extremity. Colored casting materials and polymeric casting tapes with printed decorative features are currently in demand. This has led to the development of the colored casting tapes as detailed in U.S. Pat. No. 4,699,146, and printed casting tapes described in U.S. Pat. No. 5,342,291, both incorporated herein by reference. Colored casting materials was also disclosed by Straube et al. U.S. Pat. No. 4,376,438 and in Klintworth, Jr. U.S. Pat. No. 4,934,356. The fabric of which polymeric casting tapes are made is of a material that is at least 15% extensible, and are usually made of a knitted substrate of inorganic fibers, preferably fiberglass. This fabric is then coated with the resin preferably isocyanate-functional polyurethane polymer. In creating a printed or multicolored polymeric casting tape, there are stringent dye or ink pigment requirements in order not to alter the polymerization characteristics of resin coating of the fabric which result in a shorter shelf life, and, or reduced strength. As a result, the pigments are bound to a substrate which is substantially free of those metals or metallic ions which can catalyze cure of the resin system, reactive basic groups such as tertiary amines, and of potentially reactive active hydroxy group. This is a requirement to ascertain that these casting tapes will maintain a shelf life ranging from one to five years.

Additional consideration in creating a decorative cast relates to the fact that the fabric material is stretched 15% to 20% as it is applied to an injured extremity. The results in distortion of any printed decoration as the fabric is stretched, and blurring of any decorative figure that may have been applied. Secondary considerations include detailed attention to dye pigment selection to avoid leakage of the dye and loss of decorations whenever the casting tape is soaked in water prior to application on an injured extremity. This is usually achieved by binding the dye pigment to the fabric substrate by attaching the dye pigment to a stabilizer or binder chemical without interference with the resin coating. Binder chemicals consist of acrylic and polyurethane resins, or polymers made from ethylene vinyl acetate, polyvinyl alcohol, butyl rubber, natural rubber, polyesters and mixtures of any of these materials.

Spirally striped casts have been made by concurrently winding two differently colored casting tapes, and multi-colored patterned sleeve for wearing over a cast are currently available as disclosed by Juozaitis, U.S. Pat. No. 6,047,403.

In applying these casting tapes to any parts of the body, care has to be taken by the technician or physician applying the tape to avoid staining the clothing of the patient, that of the attendant, and the walls of the casting room.

As a result of the manufacturing requirement for the production of decorative and colored casting tapes, they are generally more expensive than the plain casting tapes. This is particularly important in this era of controlled medical expenses, where the primary concern is the delivery of needed care and not fashion.

As the decorations are only needed on the most exterior surface of an applied cast, it is the object of this invention to provide an adhesive decorative art that can be applied to the exterior surface of an applied cast once the hardening process is completed.

In U.S Pat. No. 6,047,403, a decorative cast covering made of fabric and of cylindrical shape was described to be worn over an applied cast. It is more of a cylinidrical sleeve clothing which is non-adherent to a worn cast.

Decorative adhesive bandages were described in U.S. Pat. Nos. D.428,494 and D.422,083. Similar design patents were filed in the U.S. Pat. Nos. D.423,105 and D.422,708. These bandages were for application to wounds on the human skin with absorbent properties for moisture and secretions from wounds. They were all to be applied directly on to the skin and not on to another bandage or casting material. These bandages generally have an absorbent layer adherent to the inner surface of the backing. Tapes designed to adhere to skin include, for example, medical tapes such as wound or surgical dressings, athletic tapes, surgical drapes, or tapes used in adhering medical devices such as sensors, electrodes, and ostomy appliances. U.S. Pat. No. 5,614,310 suggests using a backing having a moisture transmission ability. A discontinuous adhesive coating on a breathable backing allows vapor transmission in areas of the backing not coated with adhesive. This approach was disclosed in U.S. Pat. Nos. 4,595,001, 5,613,942; EP 353972; and EP 91800. In the U.S. Pat. No. 4,285,338 Lemelson described a decorative bandage with an absorbent gauze lining, and an arcuate shaped resin shell having an illustration and also serving as a shock absorber.

In order to achieve adequate adhesive strength allow commonly available tape materials to stick to a surface, some form of priming is usually required during manufacture to improve bonding between the adhesive and and the backing material. This priming may take the form of primer coating application, surface treatments such as flame treatment and oxidizing surface treatment. This treatment may limit the porosity of the adhesive bandage. Melt additives sometimes used in film backings are not porous and may eliminate the breathability desirable of casting materials.

Even with the use of existing bandages that have breathable backing, because the skin is already covered by the multiple layers of casting materials that constitute a cured cast, any additional covering of the surface will further compromise the porosity of the applied cast. This is because the pore sizes in these bandages are relatively small and do not overlap that of an applied cast.

The need to write care instructions on an adherent tape has not been possible because paper tapes do not adhere to the uneven surface of a plaster cast. Such instructions could be as simple as to keep the involved extremity elevated, or the weight bearing status of an injured leg. The dried surface of a polymeric cast, or Plaster of Paris will not securely adhere to a pressure sensitive tape made of paper and natural and/or synthetic fabrics without developing a wrinkle. This leads to distortion of any graphics that may be applied on the outer surfaces of such adhesive tapes. As a result of this limitation, patients continue to manually paint on the surfaces of applied cast, while attempts at applying paper and fabric in my laboratory setting resulted in wrinkling and poor adhesion to plaster cast surfaces because the adhesive bearing surface of paper is completely smooth, and lacks any form of interdigitation to allow secure adherence to the rough surface of a plaster cast. Altering the strength of adhesive coating may improve adherence, but resulted in further wrinkling and distortion of applied graphic and loss of ability to removably and repeatedly apply such material to the surface of a plaster cast. The thinness of paper and standard fabric materials when used for this purpose prevents it from being easily peeled off the surface of a cast either for the purpose of change of position, or to discard it after the user has completed the use of the product.

This invention is directed a providing a pressure sensitive adhesive tape and patch made out of thick paper product, polyurethane, polyvinyl material, fabric adhered to such surfaces to achieve desired thickness, or flexible thin plastic laminate with imprinted and/or painted decorations and/or die-cut perforations on its backing. This invention is directed to a decorative pressure-sensitive adhesive tape and patch with die-cut perforation designs, imprinted graphics, or painted artwork applied to the surface of a plaster or other cast that immobilizes injured parts.

### BRIEF SUMMARY OF INVENTION

This present invention relates to an instructional and/or decorative adhesive patch and tape, made of polyurethane, polyvinyl material, thickened paper, or flexible thin plastic laminate having die-cut perforation designs, imprinted graphics, or painted designs applicable to the exterior surface of polymer casting tape, non-polymer casting tapes, and flexible bandages. It also relates to standard fabric material applied to a backing of polyurethane, polyvinyl material, thickened paper material, or any similar foam-like material. The patch form of this invention allows printed instructions relating to the care of a patient to be applied to the surface of an applied cast. This patch may also bear decorative graphics alone, or in combination with these instructions. In the tape form of this invention, the purpose is both instructional and decorative, using a one or more combinations of die-cut perforations, graphics, and words. In order to allow evaporation of moisture through an applied cast, the perforations are of a reasonable size to avoid moisture retention and skin maceration.

In the form of this invention that is used as a tape, the presence of serial perforations through the backing, and the application of adhesive and an optional underlying liner material, allows a wide area of the extremity bearing a casting material to be covered with a decorative adherent tape covering without fear of occluding the pores of the cast and increasing moisture accumulation of the underlying skin.

In another form of this invention, the pressure sensitive adhesive takes the form of a patch that can be applied in a non-continuous manner to cover part, or a wider area of the exterior surface of a plaster cast. These patches can designed in various shapes, and may combine both die-cut perforations and graphic designs on its exterior surface.

The adhesive undersurface can be made of permanently adherent pressure-sensitive adhesive commonly available, melt additives, or of an hydrogel base adhesive that can be removably applied to the exterior of an applied casting tape. These pressure-sensitive adhesive tape, fabric, polyurethane foam, polyvinyl foam, or thin plastic laminates have painted artwork, printed designs, or die-cut decorations applied to the exterior surface while the surface bearing the adhesive material is on the interior surface. The adhesive patch or tape is of a thickness that will allow easy removal by lifting it at its edges, and of a thickness that will prevent wrinkle and distortion of applied graphics.

The tape or patch of this invention can be of varying sizes for individual application over small area of an applied cast, or could be of a circumferential type encircling part or the entire surface of an applied cast. Decorative designs of varying forms, shapes, and colors are printed, painted, or die-cut on the exterior of the backing of the pressure-sensitive tape. The pressure-sensitive perforated adhesive tape and patches of this invention may have a liner applied to its innermost surface bearing the adhesive, or may be devoid of the liner material and wound on itself into a roll. This invention is directed to a decorative pressure sensitive adhesive tape and patches of a thickness that will adhere to the surface of a cast without wrinkle and without distortion of applied graphics, and in the tape form will bear serial die-cut perforations that allow evaporation of moisture through the enclosed area of skin covered by an applied casting material. This invention is further directed at a pressure sensitive tape or patches that bears instructions, words, graphics, and decorative die-cut perforations, made of a material of a thickness that will adhere to the surface of a plaster cast without wrinkle, and distortion of graphics, and can be removed at its edge and reposition without significant loss of adhesive strength.

### BRIEF DESCRIPTION OF THE DRAWINGS

FIG. 1 is a top perspective view an adhesive tape, fabric, or thin plastic laminate with multiple perforations that has been die cut through its backing.

FIG. 2 is a cross sectional view of the decorated adhesive tape or plastic laminate backing showing the two layers consisting of the optional adhesive liner and the backing bearing the adhesive on its inner surface.

### DETAILED DESCRIPTION OF THE PREFERRED EMBODIMENT OF THE INVENTION

While this invention is susceptible of embodiment in many different forms, the drawing and specification describe in detail preferred embodiments of the invention. It should be understood, however, that the disclosure is to be considered as an exemplification of the principles of the invention. The disclosure is not intended to limit the broadest aspects of the invention to the illustrated embodiments.

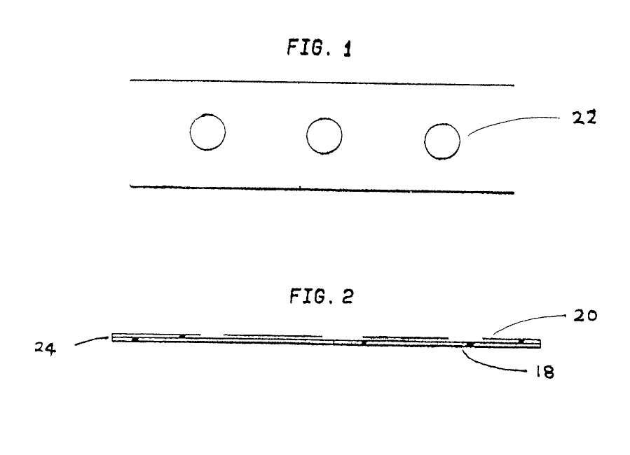

FIG. 1 is a top perspective view of an adhesive tape or thin plastic laminate with multiple perforations that has been die cut through its backing. It shows a non-adhering exterior surface of any desired shape but generally rectangular section of a pressure sensitive tape, fabric, or plastic laminate 20 that has been die cut with any desired pattern to provide a desired shape perforation 22, or imprinted with graphical designs on the exterior surface of the tape. The cross sectional view of FIG. 2 shows the relative positioning of the pressure sensitive tape, fabric, or flexible thin plastic laminate layer 20 and a removable optional protective liner 18. The pressure sensitive tape or thin plastic laminate layer 20 in one form of this invention is coextensive with a removable protective liner 18. In one embodiment of this invention, the protective liner 18 is absent, and the bandage is manufactured in a roll form allowing different dimensions of he bondage to be dispensed at different times. One or more different shaped perforations 22 are cut through the tape, fabric, or thin plastic laminate 20, with these perforations revealing the surface of a worn plaster cast when it is applied on providing decorative impressions and serving as areas of breathability. Imprinted designs, images, and color stains are also applied to the exterior surface of the pressure sensitive tape layer. These graphical images images can be applied to the exterior of the pressure sensitive tape, fabric, or thin plastic laminate layer using any of the currently available commercial printing and dyeing techniques. This decorative adhesive can also take the form patches designed in various shapes and applied to the surface of an applied cast.

When the decorative adhesive patch is considered very small, the perforations may not be necessary. Graphics imprinted on the surface patches made out of polyvinyl and polyurethane usually have to be coated superficially with a protective stain to prevent it from smudging or distoration on contact with the hand of the user. This is because the surfaces are usually shiny and does not hold the ink dye as well. Releasably bonded to adhesive bearing surface 24 of the pressure sensitive tape 20 is a removable liner or cover layer 18 which is withdrawn and discarded just before the bandage is to be used. The pressure sensitive tape is covered by adhesive on all or some of its undersurface except for the areas with through perforations. The adhesive undersurface can be made of permanently adherent pressure sensitive adhesive commonly available, melt additives, or of an hydrogel base adhesive that can be removable applied to the exterior of an applied cast. The shapes of these perforations vary, and the diameter any be non-uniform ranging from one millimeter to five centimeters depending on the size of the applied tape. The perforations may be centered on the bandage strip or positioned on the edges. Any of the commercially available medical grade synthetic and natural adhesives could be applied to the adhesive bearing surface of the tape. These include polyacrylate adhesives, polyalphaolefin adhesives, polyvinyl acrylates, rubber resin adhesives, or their mixtures. The material of the pressure sensitive tape could be of any available tape backing materials such as “MEDIPORE” and “MICROFOAM” marketed by 3-M Company, fabric materials, or any of the commercially available thin flexible plastic laminates. The backing material may also be made of a transparent material with colored imprinted designs on the exterior surface.

Generally the tape would be used with a release liner, however, a release coating or treatment could be used on the surface of the backing not provided with the pressure-sensitive adhesive tape layer to permit the tape to be wound into a roll form.

In the second form of this invention are pressure sensitive adhesive patches that are applicable to the exterior surfaces of an applied cast. The pressure sensitive patch is preferably made of a foam-like material, for example polyurethane, polyvinyl, or any other material of similar consistency. Other material option include thick paper of 1/64 thickness or more, or thin fabric applied on a backing of foam like material as stated. Such material combination is of a stiffness that it will not wrinkle, and limit distortion of any applied graphic. The preferred foam like material is polyvinyl of 1/32 inches thickness with stretch of not greater than fifteen percent. The pressure sensitive adhesive patch is coextensive with a removable protective liner. One or more different shaped perforations may be die-cut through the patch, alone or in combination with imprinted graphics. These perforations reveal the surface of a worn plaster cast when it is applied on, providing decorative impressions and serving as areas of breathability. Imprinted designs, images, and color stains are also applied to the exterior surface of the pressure sensitive adhesive patch. These graphical images can be applied to the exterior of the pressure sensitive patch by any of the currently available commercial printing and dyeing techniques. Preferably, flexographic printing technique is used when polyurethane or polyvinyl material is used. The decorative adhesive patches can be designed in various shapes and applied to the surface of an applied cast. When the decorative adhesive patch is considered very small, the perforations may not be necessary. The size of a typical adhesive patch range from ¼″ square to 5″ square, the shapes may however vary. The adhesive patches of this invention may on its exterior surface be plain colors without graphics or blank colors that could in turn be written upon with an ink pen by the user. When desired, these patches may be positioned in close proximity to each oter on the surface of a cast to form a puzzle that reveals a bigger graphical design. Instruction written on the patches are related to patients' care, but may sometimes be simple words or statement. An example is “Keep Leg Elevated”, an instruction usually given to a patient with ankle or leg injury to limit swelling.

Releasably bonded to the adhesive bearing surface of the pressure sensitive patch is a removable liner or cover layer which is withdrawn and discarded just before the patch is to be used. The pressure sensitive patch is covered by the adhesive on all or some of its undersurface except for the areas with through perforations. The adhesive undersurface can be made of permanently adherent pressure-sensitive adhesive commonly available, melt additives, or of an hydrogel base adhesive that can be removable applied to the exterior of an applied cast. The shapes of these patches vary, and the size may be non-uniform ranging from one millimeter to ten centimeters. The patch may bear perforations centered, or position elsewhere in relation to the center. Any of the commercially available medical grade synthetic and natural adhesives could be applied to the adhesive bearing surface of the tape. These include polyacrylate adhesives, polyalphaolefin adhesives, polyvinyl acrylates, rubber resin adhesives, or their mixtures. The material of the described pressure sensitive patch is preferably “MICROFOAM” marketed by 3-Company, fabric materials, or any of the commercially available thin flexible plastic laminates. The backing material may also be made of a transparent material with colored imprinted designs on the exterior surface.

What is claimed is:

1. An orthopedic cast instructional and decorative adhesive patch and tape, comprising a tape, plastic laminate, or fabric backing bearing imprinted designs and/or die cut perforations of decorative designs on its exterior surface, said decorated tape, fabric, or plastic laminate having an inner surface coated with adhesives enabling easy adherence to the surface of an applied cast.

2. The orthopedic cast instructional and decorative adhesive patch and tape, of claim 1, made of polyvinyl, polyurethane or fabric material as backing, wherein said backing has an inner surface protected by an optional removable liner.

3. The orthopedic cast decorative and instructional adhesive patch and tape of claim 1, wherein the said backing has an exterior surface optionally provided with a release coating or treatment to permit the tape to be wound into a roll form in the case of the tape form.

4. The orthopedic cast decorative and instructional adhesive patch and tape of claim 1, wherein the tape is formed as separate patches, or as a continuous tape.

5. The orthopedic cast and instructional and decorative adhesive patch tape of claim 1, wherein said backing is coated with adhesive strong enough to adhere to the surface of polymeric and non-polymeric casting materials.

6. The orthopedic cast decorative and instructional adhesive patch and tape of claim 1, wherein said backing is made of any natural, or synthetic fabric, tape material, plastic laminate, or mixture of any of these materials.

7. A method of conveying patient care related information, word instructions, and graphics on the surface of an applied cast using a pressure sensitive adhesive patch or tape of claim 1.
